(19) United States
(12) Patent Application Publication
(10) Pub. No.: US 2024/0109101 A1
(43) Pub. Date: Apr. 4, 2024

(54) METHOD FOR DRIVING A TRANSMITTING DEVICE

(71) Applicant: SUPERSONIC IMAGINE, Aix-En-Provence (FR)

(72) Inventors: Hubert CORDONNIER, Aix en Provence (FR); Vincent LAVILLE, Marseille (FR); Jean-Alain MARTIN, Aix en Provence (FR); Julien MARTINEZ, Aix-en-Provence (FR); Guillaume ROUX, Aix en Provence (FR)

(21) Appl. No.: 18/469,727

(22) Filed: Sep. 19, 2023

(30) Foreign Application Priority Data
Sep. 30, 2022 (FR) FR2210030

(51) Int. Cl. B06B 1/02; G01S 7/52

(52) U.S. Cl. CPC B06B 1/0215; G01S 7/52022; A61B 8/4488

(57) ABSTRACT

The present disclosure relates to a method for driving a transmitting device, wherein the transmitting device operates with a target frequency (f0). The method comprises: driving the transmitting device by an electrical signal comprising various drive frequencies (f1, f2) selected depending on the target frequency (f0).

### PRIOR ART

It is known to use a transmitting device, such as a transducer device comprising a plurality of transducer elements (for example disposed in an array) for communication, imaging or scanning purposes, for example in the medical imaging, radar, sonar, seismology, wireless communications, radio astronomy, acoustic and biomedecine field. An example comprises ultrasound imaging.

For this purpose, the transmitting device may operate or be driven with an electrical signal. Conventionally, said electrical signal has a predefined frequency. This frequency may for example be selected depending on the use of the transmitting device.

For example, the aim of the ultrasound imaging may be to estimate the reflectivity of a medium. Thus, the predefined frequency may be selected depending on the features of the medium (for example human tissue).

In a conventional ultrasound imaging method, an ultrasonic transducer device (also called ultrasonic probe) can be used with a set of ultrasonic transducer elements. In this method, one or more transducers are used to convert electrical energy into ultrasound waves. In particular, the transducers may transmit one or successively more ultrasound beams into a medium, which corresponds to a transmission operation. Subsequently, in a receiving operation, a set of backscattered echo signals are received from the medium by the same set or another set of transducer elements. In particular, each of the transducer elements converts an echo signal received into an electrical signal, for example. The signal can subsequently be processed by the ultrasound system or by any associated (dedicated) system. For example, they may be amplified, filtered, digitised and/or a signal conditioning operation may be carried out. The transducer elements may be disposed as a line of transducers or as an array of transducers or any other configuration.

In general, when a transmitting device operates with an electrical signal, the device transmits electromagnetic (EM) waves. The transmitting device may therefore also be called EM transmitting device.

Various standards exist that limit the authorised level of electromagnetic emissions of electrical devices, for example the standards IEC 60601-1-2, NF EN 55011/CISPR 11 and IEC 61000-4-3.

In particular, the standard CISPR 11 (see for example edition 6.2 2019-01) defines the authorised limits (that is to say the quasi-peak values of the electromagnetic emission) for various frequency ranges. Consequently, the energy of an electric field caused by a device must not exceed these limits. For example, Table 6 of the standard CISPR 11 relates to the electromagnetic radiation disturbance limits for class A group 1 equipment, measured on a test site. In addition, Table 2 of the standard CISPR 11 relates to the disturbance voltage limits for class A group 1 equipment, measured on a test site. A medical device, such as for example an ultrasonic probe, is typically of class A, because it relates to a non-domestic appliance.

Furthermore, IEC 61000-4-3/EN 61000-4-3 relates to an electromagnetic compatibility (EMC) standard, and particularly Part 4-3 addresses testing and measurement techniques, and the radiated, radio-frequency, electromagnetic field immunity test.

### DESCRIPTION OF THE DISCLOSURE

The method and the system described in the present document relate to technologies for optimising the operation or driving of a transmitting device that may improve the performances and/or the quality of the operation, despite a limited level of electromagnetic emissions caused by the transmitting device.

In particular, it may be desirable to limit the level of electromagnetic emissions in order to comply with a standard. Furthermore, it may be desirable to limit the level of electromagnetic emissions without having any impact on the operating performances desired for the transmitting device.

For example, in the case where the transmitting device is an ultrasonic probe, it may be desirable that the performance and/or acoustic energy (and/or the acoustic power) may be optimised and/or increased despite a limited level of electromagnetic emissions.

Consequently, a method for driving a transmitting device is provided according to a particular example of embodiment of the disclosure. The method comprises: driving the transmitting device by an electrical signal comprising various drive frequencies selected depending on the target frequency.

By providing such a method, it becomes possible to improve the performances and/or the quality of the operation, despite a limited level of electromagnetic emissions caused by the transmitting device.

The method simply requires a modification of the electrical signal. Therefore, the method may be easily implemented in the existing systems and transmitting devices, without requiring supplementary devices and/or structural modifications of the system or of the device. The costs for implementing the method may therefore be reduced.

Furthermore, the increase in operating performance may be obtained in any type of operating mode. Examples of various operating modes of a transmitting device in the form of an ultrasonic probe may comprise B mode (that is to say a brightness mode), a Doppler mode or a SWE mode (that is to say a shear wave elastography mode). In other terms, it is not necessary to change or to modify the specific operating mode to obtain the increase of performances, but the increase is obtained irrespectively of the selection of the operating mode.

The method according to the disclosure may include other features that may be taken separately or in combination, particularly from the following embodiments.

According to a particular example of embodiment, the target frequency corresponds to the frequency of an electrical signal that would conventionally be used to drive the transmitting device.

According to a particular example of embodiment, at least one of the drive frequencies of the electrical signal is different from the target frequency. Consequently, the drive frequencies may be different from one another.

According to a particular example of embodiment, the drive frequencies are sufficiently different and/or distant from one another such that: their totaled spectral field does not exceed the spectral field of any one of the individual drive frequencies. Consequently, the maximum defined by the total spectral field does not exceed the maxima of the spectral fields of the various frequencies.

The spectral field may be (or may comprise) an electric and/or electromagnetic field.

The totaled spectral field may consist of frequencies and of their harmonics. For example, it is possible to select the totaled spectral field such that it does not exceed the spectral field of any one of the individual drive frequencies or of their harmonics.

According to a particular example of embodiment, the drive frequencies are selected such that the target frequency can be reached by a combination of the plurality of drive frequencies.

Consequently, the various drive frequencies can be used instead of the target frequency, in order to drive the transmitting device. Consequently, the transmitting device operates as if it were driven with an electrical signal having the target frequency.

According to a particular example of embodiment, the electrical signal may consist of successive waveforms such that each waveform has for frequencies one of the drive frequencies.

A waveform within the meaning of the present disclosure may be an element of the signal in the time domain. Consequently, a waveform may also be called time component.

According to a particular example of embodiment, the waveforms are repeated (for example periodically), and/or the waveforms are alternated with one another, such that each waveform is partially interrupted.

According to a particular example of embodiment, each waveform is weighted by an impedance and/or current value depending on the target electrical signal.

According to a particular example of embodiment, the waveforms form a predefined time sequence of N1 drive frequency pulses f1, followed by N2 drive frequency pulses f2, . . . , followed by Nn drive frequency pulses fn, wherein N1, N2, Nn are whole numbers greater than or equal to 1.

However, the waveforms may also form a random time sequence of drive frequency pulses.

According to a particular example of embodiment, the waveforms and/or the drive frequency pulses are configured to stimulate a piezoelectric element (or another type of transducer element) of an ultrasonic probe (or of another type of transducer device).

According to a particular example of embodiment, the device is driven (or is configured to be driven) by an electrical signal having the target frequency, wherein the waveforms are selected, such that their set can reach (and/or approach and/or approximate) the target electrical signal. Consequently, the transmitting device may finally operate with an electrical signal adapted to the device given the target frequency of the device.

According to a particular example of embodiment, the waveforms are selected such that an average energy, referred to as “Q-Peak”, of each waveform is less than the average energy of the target electrical signal.

Therefore, the deterministic (or predefined) time sequence may for example comprise N1 frequency pulses f1, followed by N2 frequency pulses f2, followed by Nn frequency pulses fn. N1, N2, Nn may be whole numbers greater than or equal to 1 and weighted by the values of the impedances or of the currents (for example measured beforehand, optionally automatically) of the transmitting device at the frequencies f1, f2, fn, in order that the Q-Peak average energy is identical to each of these frequencies.

According to a particular example of embodiment, the method is a medical and/or ultrasound method, and/or the device is a medical and/or ultrasonic probe.

However, the transmitting device may generally be any device that operates thanks to an electrical signal and/or that transmits electromagnetic waves.

According to a particular example of embodiment, the transmitting device is driven (or is configured to be driven) by a plurality of electrical signals, each of which comprises various drive frequencies selected depending on the respective target frequency. For example, in the case where the transmitting device is a transducer device with a plurality of transducer elements, each transducer element may operate or be driven with a respective electrical signal. Consequently, the method may provide a plurality of electrical signals for a respective plurality of transducer elements.

Consequently, each of the plurality of transducer elements of the transmitting device may have a respective target frequency (for example the same target frequency).

According to a particular example of embodiment, the device transmits (or is configured to transmit) a wave to process and/or observe the medium.

According to a particular example of embodiment, the device transmits (or is configured to transmit) an electromagnetic wave when it is driven.

The present disclosure also pertains to a computer program comprising instructions that, when the program is executed by a computer, lead it to implement the method according to any one of the preceding aspects.

The present disclosure according to a particular embodiment also pertains to a system for driving a transmitting device. The transmitting device operates with a target frequency. The system is configured to: drive the transmitting device by an electrical signal comprising various drive frequencies selected depending on the target frequency.

The system may have functionalities that correspond to the operations of the method according to the present disclosure.

The features and advantages of the disclosure will become apparent upon reading the following description, given only by way of non-limiting example, and made with reference to the appended drawings. In particular, the examples illustrated in the figures may be combined unless there is significant inconsistency.

### BRIEF DESCRIPTION OF THE FIGURES

Other features and advantages of the present disclosure will become apparent upon reading the following description of particular and non-limiting examples of embodiments of the present disclosure, with reference to FIGS. 1 to 6 appended, wherein:

FIG. 1 shows a schematic drawing of a system for driving a transmitting device according to examples of the present disclosure.

FIG. 2 shows a schematic drawing of an ultrasound imaging system according to examples of the present disclosure.

FIG. 3 schematically shows a time diagram and a spectral diagram illustrating a conventional driving of a transmitting device.

FIG. 4 schematically shows a time diagram and a spectral diagram illustrating a driving of a transmitting device according to examples of the present disclosure.

FIG. 5 schematically shows a first example of relationship between drive frequencies selected and a spectral energy distribution resulting from the electric field caused by the transmitting device.

FIG. 6 schematically shows a second example of relationship between drive frequencies selected and a spectral energy distribution resulting from the electric field caused by the transmitting device according to examples of the present disclosure.

### DESCRIPTION OF THE EMBODIMENTS

In the various figures, provided by way of illustration, the same numerical references designate identical or similar elements.

FIG. 1 shows a schematic drawing of a system 10 for driving a transmitting device 20 according to examples of the present disclosure.

The system 10 may also comprise the transmitting device. Furthermore, the device may be external to the system. For example, the device 20 may be connected to the system by a cable or may communicate wirelessly. In the latter case, the device 20 may for example comprise a battery and receive a communication signal from the system that represents the electrical signal (for example the drive frequencies and/or any information included in the electrical signal). The device 20 may then generate the electrical signal internally.

The system may be a conventional system and/or the transmitting device 20 may be a conventional transmitting device. Consequently, the only difference according to the present disclosure may be the way in which the transmitting device is used by the system. However, there may be no structural differences between the system and the transmitting device according to the present disclosure and known systems and transmitting devices.

The system may be stationary or movable. The transmitting device may also be stationary or movable. For example, the system may be a fixed system (for example comprising a processing unit and a display device, as described below) and the transmitting device may be movable (for example a sensor device, a measuring device, or more particularly a probe). Nevertheless, it is also possible that the transmitting device is integrated into the system, and that the system is a movable system. For example, the system may be configured to be driven autonomously, for example thanks to an included battery. Other examples are described below.

The transmitting device may be configured to operate with an electrical signal comprising a frequency f0. Consequently, the transmitting device 20 may conventionally operate with the frequency f0.

The transmitting device 20 typically causes an electromagnetic EM emission due to its driving. Said electromagnetic (EM) emission may need to be less than a maximum EM threshold, which is for example defined by a standard.

The system is configured to drive the transmitting device 20 with an electrical signal S. The electrical signal S comprises various drive frequencies f1, f2. The drive frequencies f1, f2 are selected depending on the frequency f0. For this reason, the frequency f0 may also be called target frequency.

For example, the system may comprise a power control unit 12 configured to generate the electrical signal S. Consequently, said power control unit 12 may be configured to modulate an electrical signal so that it has various drive frequencies f1, f2 according to the present disclosure. The power control unit 12 may for example be or comprise an electronic pulser or simply “pulser” (that is to say an electronic pulse generator). The system may also comprise a processing unit 11. The processing unit may for example be configured to control the power control unit 12. For example, the processing unit 11 may compute the drive frequencies f1, f2 depending on the target frequency f0.

The processing unit may further be configured to control the transmitting device, for example by controlling the electrical signal S.

According to other examples, the system 10 may comprise at least one processing unit (or processor) 11 and in addition a memory (not shown). In examples, the processing unit and memory may be incorporated into the system as shown in FIG. 1 or may be a computer or a computing device communicatively connected thereto. According to the exact configuration and the type of computing device, the memory (which stores the instructions makes it possible to evaluate the ultrasound data or to execute the methods described in the present document) may be volatile (such as the RAM), non-volatile (such as the RAM, the flash memory, etc.) or a combination of the two. Furthermore, the system 10 may also comprise storage devices (removable and/or non-removable), including, but without being limited thereto, magnetic or optical disks or tapes. Likewise, the system 10 may also include one or more input devices such as a keyboard, a mouse, a pen, a voice input, etc. and/or one or more output devices such as a screen, loudspeakers, a printer, etc. The environment may also comprise one or more communication connections, such as LAN, WAN, point to point, etc. In certain embodiments, the connections may be used to establish point-to-point communications, connection-oriented communications, connectionless communications, etc.

The system 10 may further comprise at least one certain form of computer-readable medium. The computer-readable media may be any available medium to which the processing unit (or the processor) or other devices comprising the operating environment may access. By way of example, and without limitation, the computer-readable media may comprise computer storage media and communication media. The computer storage media comprise removable and non-removable, volatile and non-volatile media, implemented in any information storage technology or method such as computer-readable instructions, data structures, program modules or other data. The computer storage media do not comprise the communication media.

The communication media integrate computer-readable instructions, data structures, program modules or other data in a modulated data signal such as a carrier wave or another carrier mechanism, and comprise any medium providing information. The expression “modulated data signal” designates a signal of which one or more features are fixed or modified so as to code information in the signal. By way of example, and without limitation, the communication media comprise the wired media such as a wired network or a direct wired connection, and the wireless media such as the acoustic, RF, infrared, microwave media and other wireless media. The combinations of any one of the elements above must also be included in the field of application of the computer-readable media.

The system 10 may be a single computer operating in a network environment using logical connections with one or more remote computers. The remote computer may be a personal computer, a server, a router, a network PC, an approved device or another shared network node, and generally comprises a plurality or all of the elements described above as well as others not mentioned. The logical connections may include any method supported by the communication media available. Such networking environments are common in offices, corporate computer networks, intranets and the Internet.

The system 10 may be a medical system, for example an ultrasound system. Consequently, the transmitting device 20 may be a medical and/or ultrasonic probe.

For example, the system may be associated with an ultrasonic probe 20, in order to collect ultrasound data from a medium, for example living tissue and/or in particular human tissue from a person.

The ultrasonic probe 20 may comprise for example one or more transducer elements not shown in FIG. 1), each being configured to convert an electrical signal received from the system 10 into ultrasound waves. The transducer elements may be configured to transmit (a) the waves into the medium and/or to receive (b) a plurality of ultrasonic signals from the medium, optionally in response to the transmission (a). An array of transducers comprising a plurality of transducer elements may be used. The same transducer element(s) may be used to transmit a pulse and receive the response, or different transducer elements are used for the transmission and the reception. There may be one or more transmitting transducer elements and a plurality of receiving transducer elements. In another variant, a single transducer element may be used. The transducer elements may comprise piezoelectric crystals and/or other components that may be configured to generate and/or record and/or receive signals.

The ultrasonic probe 20 may further comprise an electronic control device (not shown in FIG. 1) controlling the transducer elements and acquiring signals therefrom. The electronic control device may form part of the probe. As a variant, the electronic control device may be external to the probe (for example, form part of the power control unit 12), or consist of a plurality of devices that partially form part of the probe and are partially external thereto.

Furthermore, the processing unit 11 may be configured to receive data from the transmitting device 20, process data and/or send data to an external device, such as for example, a display device, a server, a computer whereon an artificial intelligence algorithm (IA) is executed, a dedicated workstation, or any other external device.

Nevertheless, the system may be any type of electronic system. For example, the system may also be a type of medical system other than an ultrasound imaging system. Consequently, the transmitting device 20 may be any type of imaging device or sensor, using waves other than the ultrasound waves (for example, waves having a wavelength different from the wavelength of the ultrasounds and/or waves not being sound waves).

According to other examples, the system and/or the transmitting device is configured for communication, imaging or scanning purposes, for example in the medical imaging, radar, sonar, seismology, wireless communications, radio astronomy, acoustic and biomedecine field.

Examples of medical imaging systems comprise an ultrasound imaging system, an X-ray imaging system X (in particular for the mammography) and an MRI (Magnetic Resonance Imaging) system.

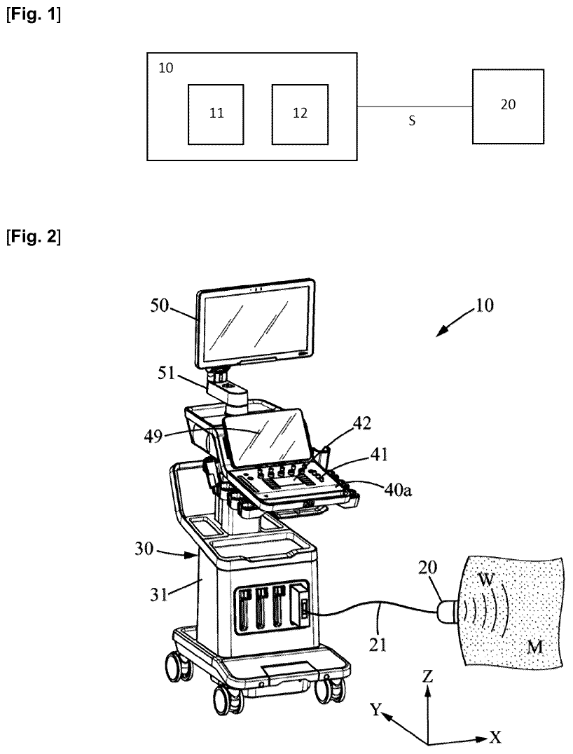

FIG. 2 shows a schematic drawing of an ultrasound imaging system 10 according to examples of the present disclosure. The ultrasound imaging system 10 may be an example of system for driving a transmitting device 10, such as described in the context of FIG. 1.

The ultrasound imaging system 10 may comprise:
a probe 20 (corresponding for example to a transmitting device 20 of FIG. 1),
a processing unit 30 for processing an image on the basis of signals received by the probe (for example corresponding to the processing unit 11 of FIG. 1),
a control panel 40 connected to the processing unit, said control panel comprising at least keys 41 and a touch pad 42, and
a screen 50 for visualising the image.

The probe 20 may be connected to the processing unit 30 by a cable 21 or by a wireless connection, and it is capable of transmitting ultrasound waves W into a medium M and of receiving ultrasound waves W from the medium M, said received ultrasound waves being consequent or resulting from reflections of said ultrasound waves transmitted on particles diffusing within said medium. The probe 20 may be an array of transducers comprising a plurality of transducers, each converting an electrical signal into a vibration and vice versa. A transducer is for example a piezoelectric element. The array of transducers may comprise one hundred transducers or more. The array of transducers is linear or curved and is disposed on an outer surface of the medium M so as to be coupled to the medium and to vibrate and to transmit or receive ultrasound waves W.

The processing unit 30 may comprise receiving devices to amplify and/or filter the signals received from the probe 20, and converters (analogue-to-digital converters and digital-to-analogue converters) to transform the signals into data representing the signal. The data may be stored in a memory of the processing unit or processed directly in order to compute intermediate processed data (beam formation data). The processing unit 30 may use any known processing method to process the image on the basis of the signals received from the probe, such as the beam formation. The processed ultrasound image data may be:
a simple image of the medium (image in B mode) generally in grey scale for visualising the organs within the medium, or
an image showing the velocity or the flow in the medium (coloured image), for example useful for visualising the blood vessels in the medium, or
an image showing a mechanical feature of the medium (elasticity), for example useful for identifying tumours within the medium (for example ShearWave™ Elastography (SWE) image data as described below).

The display screen 50 may be a screen for visualising the image processed by the processing unit 30. The display screen 50 may also visualize other information such as the scales used in the image, or configuration information for the processing or any information such as help information or a contextual help for the touch pad 42.

The display screen may be articulated on a support arm 51 for a better positioning of the user. The display screen is generally a large screen (at least 20 inches) for a better visualisation of the images by the user.

The control panel 40a is for example a portion of the housing of the system 31, said portion comprising a panel housing having a substantially flat surface inclined towards the user in order to be manipulated by a hand of said user. The control panel 40a may be moved by a hanger upwards and downwards to be adapted to the size of the user, and may optionally be moved forward and backward to be adapted to the position of the user. As can be seen in FIG. 1, the control panel 40a may comprise a display screen of the control panel 49 for visualising a plurality of configuration information. This display screen of the control panel 49 may also be articulated on the housing of the control panel 48 to be inclined in an inclination different from that of the surface of the control panel.

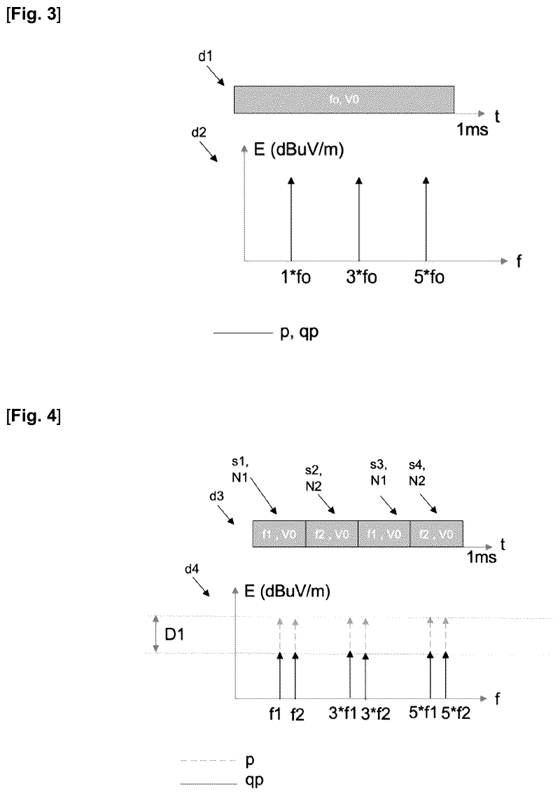

FIG. 3 schematically shows a time diagram and a spectral diagram illustrating a conventional driving of a transmitting device. In particular, the diagram d1 shows a time diagram of a conventional electrical signal for driving a transmitting device, and the diagram d2 shows the respective spectral diagram of the conventional electrical signal.

As shown in the time diagram d1, a transmitting device can operate conventionally with an electrical signal comprising a single frequency f0 throughout the entire duration of the driving. The frequency may be selected depending on the objective of the transmitting device. For example, in the case where the transmitting device is an ultrasonic probe for the imaging of a medium, the frequency f0 may be selected to correspond to the features of the medium. For example, the frequency may be f0=5 MHz.

For example, a frequency of approximately 5 MHz (or any value between for example 1 and 20 MHz) may be selected to generate ultrasound waves for the imaging of human or animal tissue. This value may be advantageous for this application, because the attenuation caused by the tissue in this frequency range is relatively low and, at the same time, the ultrasound waves in this range may be easily generated by a piezoelectric element of an ultrasonic transducer element. Thus, the low-frequency ultrasound waves (in the useful frequency band of piezoelectric elements) may penetrate more deeply into the tissues. The selection of such a frequency (5 MHz for example) may therefore be a compromise between the penetration depth and the electro-acoustic efficiency of the piezoelectric element.

In the example of the diagram d1, the drive time is of 1 ms. Nevertheless, the drive time may also be longer or shorter. The drive time may for example correspond to the time for transmitting an ultrasound pulse (transmitted by the transmitting device). In addition, the electrical signal may have a voltage of, for example, VO=50 V.

The corresponding spectral diagram d2 may indicate the electric field (dBuV/m) (and consequently the power and/or energy) transmitted by the transmitting device depending on the frequency (f). As presented in the spectral diagram d2, the electrical signal may comprise a frequency f0 and harmonics 3*f0, 5*f0, etc. An instantaneous energy and/or power p (that is to say a peak power) and an average energy and/or power qp (that is to say a “quasi-peak”) over the total drive duration are similar. It should be noted that in the present disclosure, “average energy” and “power” may be used interchangeably, because the parameters may correspond to one another, by taking into account a particular time (for example a pulse of 1 ms).

For example, this particular time to be taken into account may be 1 ms in the examples of FIGS. 3 and 4. Thus, in the example of FIG. 4, the average energy transmitted by the transmitting device in a single drive frequency (for example f1) is reduced, because the total time of the respective wavelengths (for example S1 and S3) is shorter than the total time taken into account (for example 1 ms).

The electromagnetic emission caused by the transmitting device due to its driving may be defined by the average energy and/or power. Consequently, a standard that governs the authorised level of electromagnetic emission may require limiting the average energy and/or power. Consequently, in a conventional driving method such as illustrated in FIG. 3, the instantaneous energy and/or power must also be limited, respectively.

In the case of the example mentioned where an ultrasonic probe operates with an electrical signal having a frequency of f0=5 MHz, and where harmonics of 25 MHz are also considered, the authorised electromagnetic radiation quasi-peak may be limited to 40 dB (see for example Table 6 of the standard CISPR 11 mentioned above). Beyond 230 MHz, the authorised quasi-peak may be limited to 47 dB. The electromagnetic radiation of an ultrasonic probe must therefore be located below these upper limits to meet in particular these standards.

FIG. 4 schematically shows a time diagram and a spectral diagram illustrating a driving of a transmitting device according to examples of the present disclosure.

The diagrams d3 and d4 mainly correspond to the diagrams d1 and d2 of FIG. 3. However, the method according to the present disclosure is applied to drive the transmitting device. In particular, the diagram d3 shows a time diagram of an electrical signal for driving a transmitting device according to examples of the present disclosure, and the diagram d4 shows the respective spectral diagram of the electrical signal according to examples of the present disclosure.

As shown in the time diagram d3, a transmitting device is driven with an electrical signal comprising various drive frequencies f1, f2. More than two drive frequencies may also be used. The drive frequencies are selected depending on the target frequency f0. The target frequency may be the frequency f0 used in the example of FIG. 3. For example, the frequency may be f0=5 MHz. Consequently, the frequency f1 may be for example f1=4.5 MHz and/or the frequency f2 may be for example f2=5.5 MHz. The electrical signal may have a voltage of for example of VO=50 V.

Generally, at least one of the drive frequencies f1, f2 of the electrical signal may be different from the target frequency. The drive frequencies are preferably selected such that the target frequency can be reached by a combination of the plurality of drive frequencies.

Consequently, the various drive frequencies can be used instead of the target frequency, in order to drive the transmitting device. Consequently, the transmitting device operates as if it were driven with an electrical signal having the target frequency.

The electrical signal may consist of successive waveforms S1, S2, S3, S4, . . . , such that each waveform has for frequencies one of the drive frequencies f1, f2.

The waveform may be an element of the signal in the time domain. Consequently, the waveform may also be called time component.

As indicated in the example of FIG. 4, the waveforms relating to the frequencies may be repeated, for example periodically. Furthermore, the waveforms may be alternated with one another, so that each waveform is partially interrupted.

In particular, the waveforms may form a predefined time sequence of N1 drive frequency pulses f1, followed by N2 drive frequency pulses f2, . . . , followed by Nn drive frequency pulses fn, N1, N2, Nn being whole numbers greater than or equal to 1.

However, the waveforms may also form a random time sequence of drive frequency pulses.

Each waveform may be weighted by an impedance and/or current value depending on the target electrical signal.

In the example of the diagram d3, the drive time is of 1 ms. Nevertheless, the drive time may also be longer or shorter. The drive time may for example correspond to the time for transmitting an ultrasound pulse (transmitted by the transmitting device).

As shown in the corresponding spectral diagram d4, the electrical signal may comprise the frequencies f1, f2 and the harmonics 3*f1, 3*f2, 5*f1, 5*f2, . . . the average energy and/or power qp (that is to say a “quasi-peak”) over the complete drive time (for example 1 ms) may be less than the instantaneous energy and/or power p (that is to say a power peak). This difference may be due to time intervals (indicated in the diagram d3), for which a specific waveform (for example of frequency f1) is not used and therefore not transmitted by the transmitting device.

This difference may apply to the frequencies f1 and f2, and optionally also to one or more of their harmonics.

In particular, the waveforms (illustrated in Diagram d3) may be selected such that an average energy and/or power (Q-Peak) of each waveform is less than the average energy and/or power of the target electrical signal.

Consequently, given that the electromagnetic emission caused by the transmitting device may be defined by the average energy and/or power, the electromagnetic emission may be reduced, even if the instantaneous energy and/or power is higher.

For example, a standard that governs the authorised level of electromagnetic emission may be complied with due to the limited average energy and/or power.

Consequently, according to the present disclosure, the instantaneous energy and/or power does not need to be limited to be below this limit. Consequently, the performances of the transmitting device may be advantageously increased, while respecting the limitations required by the standard(s).

In one example, the difference D1 between the instantaneous energy and/or power and the average energy and/or power may be D1=20*log(2)=6 dB.

An operating mode of a transmitting device in the form of an ultrasonic probe may comprise for example a ShearWave™, referred to as SWE, mode. This mode comprises an excitation step during which a shear wave is generated in the medium, as described for example in the patent EP 1 546 757 B1 by the same applicant. During the excitation step (a), the elastic shear wave is generated thanks to transmitting at least one sufficiently powerful focused ultrasound wave (PUSH) into the medium. For example, a focused ultrasound wave (PUSH) may correspond to a waveform according to the present disclosure.

The emission sequences used for the PUSH may consist of thousands of square wave signal periods at a given frequency. According to the present disclosure, it is advantageously possible to alternatively use a square wave signal period with a 1st frequency f1 and a square wave signal period with a 2nd frequency f2, which makes it possible to reduce the peaks of the frequency spectrum during electromagnetic emission measurements (for example of the electromagnetic compatibility (EMC) and of electromagnetic interference (EMI)).

An elaborate version of the present disclosure may use any number of central frequencies, randomly time-sequenced, with a time and/or amplitude (voltage, cyclic ratio, etc.) weighting, in any imaging mode and for any ultrasonic probe, which makes it possible to expand the frequency spectrum transmitted during the electromagnetic emission measurements. The random sequence of frequencies makes it possible to reduce the electrical noises intrinsic to the system and therefore make it possible to improve the image quality. The weighting makes it possible to optimise the amplitude of the peaks of the frequency spectrum transmitted during electromagnetic emission measurements, so as to maintain a margin in relation to the limits of a standard, and this in the entire frequency band.

Consequently, a first variant according to one example of the present disclosure may use any number of central frequencies, deterministically time-sequenced, in any imaging mode and for any ultrasonic probe, which makes it possible to reduce the peaks of the frequency spectrum during measurements referred to as EMC/EMI for validating systems in relation to the standards

A second variant according to one example of the present disclosure may use any number of central frequencies, randomly time-sequenced, in any imaging mode and for any ultrasonic probe, which makes it possible to reduce the peaks of the frequency spectrum during EMC/EMI measurements. The random sequence of frequencies makes it possible to reduce the electrical noises intrinsic to the system and therefore make it possible to improve the image quality.

A third variant according to one example of the present disclosure may use any number of central frequencies, time-sequenced, with a time and/or amplitude (voltage, cyclic ratio, etc.) weighting, in any imaging mode and for any ultrasonic probe, which makes it possible to expand the frequency spectrum transmitted during the EMC/EMI measurements. The weighting makes it possible to optimise the amplitude of the peaks of the frequency spectrum transmitted during EMC/EMI measurements, so as to maintain a margin in relation to the limits of EMC/EMI standards, in the entire frequency band.

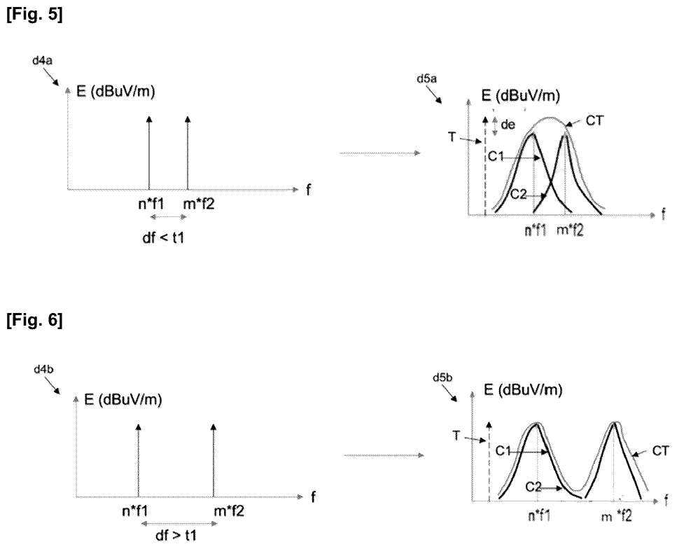

FIG. 5 schematically shows a first example of relationship between drive frequencies selected and a spectral energy distribution resulting from the electric field (and therefore from the electromagnetic emission) caused by the transmitting device.

The diagram d4a mainly corresponds to the diagram d3 of FIG. 4. Consequently, the electrical signal may comprise the frequencies f1, f2 and the harmonics 3*f1, 3*f2, 5*f1, 5*f2, etc. To simplify the schematic illustration of FIG. 5, the frequencies are indicated by n*f1 and m*f2, where n=1, 2, 3, . . . and/or m=1, 2, 3, . . .

The diagram d5a schematically shows a spectral energy distribution resulting from the electromagnetic emission of the transmitting device. In particular, the diagram d5a shows the overall electric field (that is to say the totaled field) CT resulting from the sum of the electric fields C1 of frequency n*f1 and C2 of m*f2.

As shown in diagram d5a, the difference between the frequencies n*f1 and m*f2 may influence the maximum T of the total field (that is to say the totaled field) CT and therefore the respective average energy and/or power.

In the example of FIG. 5, the distance df between the frequencies n*f1 and m*f2 may be below a threshold t1. Thus, the frequencies n*f1 and m*f2 may be so close that the maximum T of the total field (that is to say the totaled sum) CT exceeds the maxima of the electric fields C1 and C2 of a quantity de. Such a selection of frequencies f1 and f2 is therefore less desirable, because the effect sought of reducing the average energy and/or power may not be achieved or is at least lessened.

FIG. 6 schematically shows a second example of relationship between drive frequencies selected and a spectral energy distribution resulting from the electric field and (therefore from the electromagnetic emission) caused by the transmitting device according to examples of the present disclosure.

The diagrams d4b and d5b of FIG. 6 mainly correspond to the diagrams d4a and d5a of FIG. 6. Nevertheless, in the example of FIG. 6, the frequencies n*f1 and m*f2 are sufficiently distant so that the maximum T of the total field (that is to say the totaled field) CT does not exceed (or at least not substantially) the maxima of the electric fields C1 and C2. Such a selection of frequencies f1 and f2 is therefore desirable, because the effect sought of reducing the average energy and/or power may be achieved.

Consequently, the difference df between the frequencies f1 and f2 may exceed the minimal threshold t1.

For example, the minimal threshold t1 may be selected depending on a distribution of the total electric field, particularly such that the maximum of the total electric field does not exceed the maxima of the electric fields related to the frequencies f1, f2.

Therefore, the drive frequencies may be sufficiently different and/or distant from one another such that their totaled spectral field does not exceed the spectral field of any one of the individual drive frequencies.

Consequently, the desired effect of reducing the average energy and/or power may advantageously be achieved.

All of these embodiments and other examples such as described above are given purely by way of non-limiting examples, and may be combined and/or modified according to the scope of the present disclosure.

1. A method for driving a transmitting device, wherein the transmitting device operates with a target frequency, the method comprising:
driving the transmitting device by an electrical signal comprising various drive frequencies selected depending on the target frequency.

2. The method according to claim 1, wherein at least one of the drive frequencies of the electrical signal is different from the target frequency.

3. The method according to claim 1, wherein the drive frequencies are sufficiently one of different and distant from one another such that:
a total spectral field thereof does not exceed the spectral field of any one of the individual drive frequencies.

4. The method according to one of the preceding claim 1, wherein
the drive frequencies are selected such that the target frequency can be reached by a combination of the plurality of drive frequencies.

5. The method according to claim 1, wherein the electrical signal consists of successive waveforms such that each waveform has for frequency one of the drive frequencies.

6. The method according to claim 5, wherein at least one of:
the waveforms are repeated, and
the waveforms are alternated with one another, so that each waveform is partially interrupted.

7. The method according to claim 5, wherein each waveform is weighted by at least one of an impedance and a current value depending on the target electrical signal.

8. The method according to claim 5, wherein the waveforms form a predefined time sequence of N1 drive frequency pulses f1, followed by N2 drive frequency pulses f2, . . . , followed by Nn drive frequency pulses fn;
N1, N2, Nn being whole numbers greater than or equal to 1.

9. The method according to claim 5, wherein the waveforms form a random time sequence of drive frequency pulses.

10. The method according to claim 8, wherein at least one of the waveforms and the drive frequency pulses are configured to stimulate a piezoelectric element of an ultrasonic probe.

11. The method according to claim 5, wherein the device is controlled by an electrical signal having the target frequency, wherein the waveforms are selected, such that their set can reach the target electrical signal.

12. The method according to claim 5, wherein the waveforms are selected such that an average energy, referred to as “Q-Peak”, of each waveform is less than the average energy of the target electrical signal.

13. The method according to claim 1, wherein at least one of:
the method is at least one of a medical and an ultrasound method; and
the device is at least one of a medical and an ultrasonic probe.

14. The method according to claim 1, wherein at least one of:
the transmitting device transmits a wave in order to at least one of process and observe the medium, and
the device transmits an electromagnetic wave when the device is driven.

15. A computer program comprising instructions that, when the program is executed by a computer, implements the method according to claim 1.

16. A system for driving a transmitting device, wherein the transmitting device operates with a target frequency, the system being configured to:
drive the transmitting device by an electrical signal comprising various drive frequencies selected depending on the target frequency.
